(19) United States
(12) Patent Application Publication
(10) Pub. No.: US 2021/0045993 A1
(43) Pub. Date: Feb. 18, 2021

(54) COSMETIC COMPOSITIONS

(71) Applicant: Eleblend LLC, West Bridgewater, MA (US)

(72) Inventors: Gerald S. Jones, Jr., Norwood, MA (US); Joseph P. St. Laurent, Lakeville, MA (US); Scott A. Goodrich, Stoughton, MA (US)

(21) Appl. No.: 17/085,166

(22) Filed: Oct. 30, 2020

Related U.S. Application Data
(63) Continuation of application No. PCT/US2019/030011, filed on Apr. 30, 2019.
(60) Provisional application No. 62/664,766, filed on Apr. 30, 2018.

(51) Int. Cl. A61K 8/891; A61K 8/34; A61K 31/05; A61K 8/36; A61K 8/37; A61K 8/25; A61K 8/92

(52) U.S. Cl. CPC A61K 8/891; A61K 8/342; A61K 31/05; A61K 2800/48; A61K 8/37; A61K 8/25; A61K 8/922; A61K 8/361

(57) ABSTRACT

The present invention relates to a composition such as an anhydrous gel, and its use for example as a cosmetic base, comprising a silicone-based excipient and one or more carrier oils, and methods of administering the composition to a subject.

### CROSS-REFERENCE TO RELATED APPLICATIONS

This application is a continuation of International Application Number PCT/US2019/030011 filed Apr. 30, 2019, which claims priority to U.S. Ser. No. 62/664,766 filed Apr. 30, 2018, the contents of which are incorporated herein by reference in their entirety.

### BACKGROUND

Cosmetic bases are prepared to accept the addition of a variety of components such as carrier oils, essential oils, functional excipients, and therapeutic skincare products, enabling a formulator to create personalized blends with minimal effort. They can also be used on their own for simple, gentle skincare. Most commercially available cosmetic bases are examples of oil-in-water and water-in-oil emulsions, which are inappropriate for the inclusion of moisture-sensitive materials. Care must be taken when adding components to water-containing bases such that the component to be added is compatible with other formulation or aqueous ingredients, and that the integrity of the emulsion is maintained. These water-based emulsions are prone to freeze-thaw cycles that can destroy the emulsion resulting in phase separation and loss of utility. Water-containing bases also require the inclusion of preservatives and microbicides to prolong shelf-life and prevent bacterial/fungal colonization.

Anhydrous cosmetic bases are characterized as water-free and, therefore, free of limitations imposed by water regarding inclusion of moisture-sensitive materials and influence on physical properties of ionizable formulation ingredients (acids and bases). Anhydrous bases are typically more compatible with a wider range of organic ingredients and lipophilic actives, and possess intrinsically longer shelf-lives without need of preservatives and microbicides. There is need for anhydrous cosmetic bases that can be used for the incorporation of diverse organic ingredients, and which are free of the limitations imposed by the presence of water.

### SUMMARY

The present invention relates to a composition such as an anhydrous gel, and its use for example as a cosmetic base, comprising a silicone-based excipient, e.g., a silicone elastomer, and one or more carrier oils, e.g., one or more natural oils, and methods of administering the composition to a subject, e.g., by topical or transdermal administration.

In one embodiment, the composition is an anhydrous gel. In another embodiment, the anhydrous gel comprises one or more volatile aliphatic hydrocarbons, one or more liquid fatty esters, one or more lactate esters, one or more particular essential oils, and one or more silicone fluids. In another embodiment, the weight ratio of silicone elastomer to the one or more oils is about 1.5-to-0.5. In another embodiment, the composition comprises cannabis, a product derived from cannabis, e.g., hemp oil, hemp seed oil, or cannabis oil, or a cannabinoid.

In one aspect, described herein is a composition comprising a silicone-based excipient, a carrier oil, an emollient, and a viscosity enhancement agent, wherein the composition does not comprise a healthcare active. In some embodiments, the viscosity enhancement agent is silica siliylate. In some embodiments, the silicone-based excipient is present in the composition in at least about 80% by weight. In some embodiments, the a silicone-based excipient is a silicone elastomer. In some embodiments, the silicone elastomer is provided in a carrier fluid. In some embodiments, the silicone elastomer is dimethicone/bis-isobutyl polypropylene glycol crosspolymer, e.g., dimethicone/bis-isobutyl PPG-20 crosspolymer. In some embodiments, the carrier fluid is isododecane. In some embodiments, the weight ratio of carrier fluid to silicone elastomer is about 85:15. In some embodiments, the carrier oil is a natural oil. In some embodiments, the carrier oil is selected from the group consisting of sunflower seed oil, coconut oil, grape seed oil, olive oil, jojoba, and combinations thereof. In some embodiments, the carrier oil is sunflower seed oil. In some embodiments, the carrier oil is present in the composition in at least about 5% by weight, e.g., about 5%, about 10%, about 20%, or about 30% by weight. In some embodiments, the weight ratio of silicone elastomer to carrier oil is from about to about 1:0.2 to about 2:1. In some embodiments, the weight ratio of silicone elastomer to carrier oil is about 1.5:0.5. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition further comprises a silicone fluid. In some embodiments, the silicone fluid is a trimethicone. In some embodiments, the trimethicone is phenyl trimethicone. In some embodiments, the composition is an anhydrous gel. In some embodiments, the composition comprises less than 0.5% of water by weight. In some embodiments, the composition further comprises an additional essential oil. In some embodiments, the essential oil is selected from the group consisting of birch oil, lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1% by weight, e.g., 1%, 3%, 4%, 5%, 10%, 20%, or 30% by weight. In some embodiments, the composition further comprises a terpenoid, e.g., camphor. In some embodiments, the composition further comprises a wax, e.g., beeswax. In some embodiments, the composition further comprises cannabis or a product derived from cannabis. e.g., a product derived from cannabis such as hemp oil, hemp seed oil, or cannabis oil. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In one aspect, described herein is a composition comprising a silicone-based excipient, a carrier oil, an emollient, and a viscosity enhancement agent, wherein the composition does not comprise lidocaine, fluocinonide, or tacrolimus. In some embodiments, the viscosity enhancement agent is silica siliylate. In some embodiments, the silicone-based excipient is present in the composition in at least about 80% by weight. In some embodiments, the a silicone-based excipient is a silicone elastomer. In some embodiments, the silicone elastomer is provided in a carrier fluid. In some embodiments, the silicone elastomer is dimethicone/bis-isobutyl polypropylene glycol crosspolymer, e.g., dimethicone/bis-isobutyl PPG-20 crosspolymer. In some embodiments, the carrier fluid is isododecane. In some embodiments, the weight ratio of carrier fluid to silicone elastomer is about 85:15. In some embodiments, the carrier oil is a natural oil. In some embodiments, the carrier oil is selected from the group consisting of sunflower seed oil, coconut oil, grape seed oil, olive oil, jojoba, and combinations thereof. In some embodiments, the carrier oil is sunflower seed oil. In some embodiments, the carrier oil is present in the composition in at least about 5% by weight, e.g., about 5%, about 10%, about 20%, or about 30% by weight. In some embodiments, the weight ratio of silicone elastomer to carrier oil is from about to about 1:0.2 to about 2:1. In some embodiments, the weight ratio of silicone elastomer to carrier oil is about 1.5:0.5. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition further comprises a silicone fluid. In some embodiments, the silicone fluid is a trimethicone. In some embodiments, the trimethicone is phenyl trimethicone. In some embodiments, the composition is an anhydrous gel. In some embodiments, the composition comprises less than 0.5% of water by weight. In some embodiments, the composition further comprises an additional essential oil. In some embodiments, the essential oil is selected from the group consisting of birch oil, lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1%, e.g., 1%, 3%, 4%, 5%, 10%, 20%, or 30% by weight. In some embodiments, the composition further comprises cannabis or a product derived from cannabis. In some embodiments, the product derived from cannabis is selected from the group consisting of cannabis oil, hemp oil, and hemp seed oil. In some embodiments, the composition further comprises a cannabinoid, e.g., a cannabinoid described herein. In some embodiments, the cannabinoid is derived from a naturally occurring source. In some embodiments, the cannabinoid is a synthetic cannabinoid. In some embodiments, the composition further comprises a terpenoid, e.g., camphor. In some embodiments, the composition further comprises a wax, e.g., beeswax. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In another aspect, described herein is a composition formulated for topical or transdermal delivery, the composition comprising a cannabinoid, a carrier oil, a silicone elastomer, and a viscosity enhancement agent, e.g., silica silylate. In some embodiments, the composition is anhydrous, e.g., comprising less than 0.5% of water by weight. In some embodiments, the composition is an anhydrous gel. In some embodiments, the silicone-based excipient is present in the composition in at least about 80% by weight. In some embodiments, the silicone elastomer is dimethicone/bis-isobutyl polypropylene glycol crosspolymer, e.g., dimethicone/bis-isobutyl PPG-20 crosspolymer. In some embodiments, the silicone elastomer is provided in a carrier fluid. In some embodiments, the carrier fluid is isododecane. In some embodiments, the weight ratio of carrier fluid to silicone elastomer is about 85:15. In some embodiments, the composition further comprises an oil, e.g., a natural oil, e.g., sunflower seed oil, peppermint oil, eucalyptus oil, cannabis oil, or a combination thereof. In some embodiments, the weight ratio of silicone elastomer to oil is from about to about 1:0.2 to about 2:1. In some embodiments, the weight ratio of silicone elastomer to oil is about 1.5:0.5. In some embodiments, the composition comprises an ester. In some embodiments, the ester is a lactate ester. In some embodiments, the lactate ester is lauryl lactate. In some embodiments, the ester is present in the composition in an amount of less than about 0.5% by weight. In some embodiments, the ester is present in the composition in an amount of from about 0.5% to about 5% by weight. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition is substantially free of an antimicrobial. In some embodiments, the composition further comprises a silicone fluid. In some embodiments, the silicone fluid comprises a trimethicone. In some embodiments, the trimethicone is phenyl trimethicone. In some embodiments, the cannabinoid is derived from a naturally occurring source, e.g., a natural oil such as cannabis oil, hemp oil, and hemp seed oil. In some embodiments, the cannabinoid is a synthetic cannabinoid. In some embodiments, the cannabinoid is a cannabinoid described herein. In some embodiments, the composition comprises a plurality of cannabinoids. In some embodiments, the plurality of cannabinoids comprises cannabidiol and tetrahydrocannabinol. In some embodiments, the cannabidiol comprises a synthetic cannabidiol. In some embodiments, the cannabinoid is a cannabinoid described herein. In some embodiments, the composition does not comprise diethylene glycol monoethyl ether. In some embodiments, the composition further comprises an additional essential oil. In some embodiments, the essential oil is selected from the group consisting of birch oil, lavender oil, eucalyptus oil, peppermint oil, a high terpene oil, and combinations thereof. In some embodiments, the essential oil is selected from the group consisting of lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1% by weight, e.g., 1%, 3%, 4%, 5%, 10%, 20%, or 30% by weight. In some embodiments, the composition is substantially free of an antimicrobial. In some embodiments, the composition does not comprise diethylene glycol monoethyl ether. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In another aspect, described herein is a composition, the composition comprising a carrier oil, an essential oil, an emollient, a silicone elastomer, and a cannabinoid. In some embodiments, the composition further comprises a viscosity enhancement agent, e.g., silica silylate. In some embodiments, the composition is anhydrous, e.g., comprising less than 0.5% of water by weight. In some embodiments, the composition is an anhydrous gel. In some embodiments, the silicone-based excipient is present in the composition in at least about 80% by weight. In some embodiments, the silicone elastomer is dimethicone/bis-isobutyl polypropylene glycol crosspolymer, e.g., dimethicone/bis-isobutyl PPG-20 crosspolymer. In some embodiments, the silicone elastomer is provided in a carrier fluid. In some embodiments, the carrier fluid is isododecane. In some embodiments, the weight ratio of carrier fluid to silicone elastomer is about 85:15. In some embodiments, the composition further comprises an oil, e.g., a natural oil, e.g., sunflower seed oil, peppermint oil, eucalyptus oil, cannabis oil, or a combination thereof. In some embodiments, the weight ratio of silicone elastomer to oil is from about to about 1:0.2 to about 2:1. In some embodiments, the weight ratio of silicone elastomer to oil is about 1.5:0.5. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition is substantially free of an antimicrobial. In some embodiments, the composition further comprises a silicone fluid. In some embodiments, the silicone fluid comprises a trimethicone. In some embodiments, the trimethicone is phenyl trimethicone. In some embodiments, the composition does not comprise diethylene glycol monoethyl ether. In some embodiments, the essential oil is selected from the group consisting of birch oil, lavender oil, eucalyptus oil, peppermint oil, a high terpene oil, and combinations thereof. In some embodiments, the essential oil is selected from the group consisting of lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1% by weight, e.g., 1%, 3%, 4%, 5%, 10%, 20%, or 30% by weight. In some embodiments, the composition further comprises cannabis or a product derived from cannabis. In some embodiments, the cannabinoid is a cannabinoid described herein. In some embodiments, the composition comprises a plurality of cannabinoids, e.g., a plurality of cannabinoids described herein. In some embodiments, the cannabinoid is derived from a naturally occurring source, e.g., cannabis oil, hemp oil, and hemp seed oil. In some embodiments, the cannabinoid is a synthetic cannabinoid. In some embodiments, the composition is substantially free of an antimicrobial. In some embodiments, the composition does not comprise diethylene glycol monoethyl ether. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In an aspect, described herein is a method of adding an additional component to a composition, e.g., a composition formulated for topical or transdermal administration, comprising a silicone-based excipient, a carrier oil, an emollient, and a viscosity enhancement agent. In some embodiments, the additional component is an essential oil. In some embodiments, the essential oil is selected from the group consisting of birch oil, lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is added to the composition in an amount that is at least about 1%, e.g., 1%, 3%, 4%, 5%, 10%, 20%, or 30%, of the total weight of the composition. In some embodiments, the additional component comprises cannabis or a product derived from cannabis. In some embodiments, the product derived from cannabis is hemp oil, hemp seed oil, or cannabis oil. In some embodiments, the additional component comprises a cannabinoid, e.g., a cannabinoid described herein. In some embodiments, the additional component comprises a plurality of cannabinoids, e.g., a plurality of cannabinoids described herein. In some embodiments, the plurality of cannabinoids comprises cannabidiol and tetrahydrocannabinol. In some embodiments, the cannabidiol comprises a synthetic cannabidiol. In some embodiments, the cannabidiol is derived from a natural oil, e.g., cannabis oil, hemp oil, or hemp seed oil. In some embodiments, the additional component comprises an essential oil, e.g., an essential oil described herein, and a cannabinoid, e.g., a cannabinoid described herein. In some embodiments, the additional component comprises an essential oil, e.g., an essential oil described herein, and cannabis or a product derived from cannabis, e.g., a product derived from cannabis such as hemp oil, hemp seed oil, or cannabis oil. In some embodiments the cannabinoid is derived from a naturally-occurring source, e.g., a natural oil such as hemp oil, hemp seed oil, or cannabis oil. In some embodiments, the essential oil is present in the composition in at least about 1% by weight, e.g., 1%, 3%, 4%, 5%, 10%, 20%, or 30% by weight. In some embodiments, the cannabinoid is present in the composition in at least about 1% by weight, e.g., 1%, 3%, 4%, 5%, 10%, 20%, or 30% by weight. In some embodiments, the additional component comprises a terpenoid, e.g., camphor. In some embodiments, the composition is administered on a keratinous surface of the subject. In some embodiments, the keratinous surface is the skin of the subject. In some embodiments, the subject is a mammal. In some embodiments, the mammal is a human.

### DETAILED DESCRIPTION

### Definitions

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 carbon atoms in it. The term “haloalkyl” refers to an alkyl in which one or more hydrogen atoms are replaced by halo, and includes alkyl moieties in which all hydrogens have been replaced by halo, e.g., perfluoroalkyl. The terms “arylalkyl” or “aralkyl” refer to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

The term “alkylene” refers to a divalent alkyl, e.g., —CH₂—, —CH₂CH₂—, and —CH₂CH₂CH₂—.

The term “alkenyl” refers to a straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkynyl” refers to a straight or branched hydrocarbon chain containing 2-12 carbon atoms and characterized in having one or more triple bonds. Examples of alkynyl groups include, but are not limited to, ethynyl, propargyl, and 3 -hexynyl. One of the triple bond carbons may optionally be the point of attachment of the alkynyl substituent.

The term “aryl” refers to an aromatic monocyclic, bicyclic, or tricyclic hydrocarbon ring system, wherein any ring atom capable of substitution can be substituted, e.g., by one or more substituents. Examples of aryl moieties include, but are not limited to, phenyl, naphthyl, and anthracenyl.

The term “arylalkyl” or the term “aralkyl” refers to alkyl substituted with an aryl. Exemplary aralkyls include but are not limited to benzyl and phenethyl.

The term “cycloalkyl” as employed herein includes saturated cyclic, bicyclic, tricyclic, or polycyclic hydrocarbon groups having 3 to 12 carbons. Any ring atom can be substituted, e.g., by one or more substituents. The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbornyl.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer.

ee=(90−10)/100=80%.

Thus, a composition containing 90% of one enantiomer and 10% of the other enantiomer is said to have an enantiomeric excess of 80%. Some of the compositions described herein contain an enantiomeric excess of at least 50%, 75%, 90%, 95%, or 99% of Compound 1 (the S-enantiomer). In other words the compositions contain an enantiomeric excess of the S enantiomer over the R enantiomer.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, Si, P or S, e.g., carbon atoms and 1-3, 1-6, or 1-9 heteroatoms of N, O, Si, P or S if monocyclic, bicyclic, or tricyclic, respectively. The heteroatom may optionally be the point of attachment of the heterocyclyl substituent. Any ring atom can be substituted, e.g., by one or more substituents. The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl, pyrimidinyl, quinolinyl, and pyrrolidinyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, Si, P or S, e.g., carbon atoms and 1-3, 1-6, or 1-9 heteroatoms of N, O, Si, P or S if monocyclic, bicyclic, or tricyclic, respectively. Any ring atom can be substituted, e.g., by one or more substituents.

As used herein, the term “about” denotes an approximate range of plus or minus 10% from a specified value. For instance, the language “about 20%” encompasses a range of 18-22%. As used herein, “about” also includes the exact amount. Hence “about 20%” means “about 20%” and also “20%.”

As used herein, “anhydrous” means that, other than water of hydration contained in the various components used to formulate the product, no free water is added to the composition. Typically, the water content of an anhydrous composition described herein will be less than 5% by weight. Preferably the water content of the composition is less than 3% and most preferably less than about 1% by weight of the composition.

A “keratinous substrate”, as used herein, includes but is not limited to, skin, hair, eyelashes, lips and nails.

As used herein, “emollient” is a hydrophobic material that provides softness, lubricity and smoothness to the skin and often forms a thin occlusive film which increases hydration by reducing transepidermal water loss (TEWL).

As used herein, “polymer polydispersity index (PDI)” or “polymer polydispersity” refers to the distribution of molecular mass in a given polymer sample. The polymer PDI calculated is the weight average molecular weight divided by the number average molecular weight. It indicates the distribution of individual molecular masses in a batch of polymers. The polymer PDI has a value typically greater than 1, but as the polymer chains approach uniform chain length, the PDI approaches unity (1).

The term “substantially free” when referring to a compound or composition described herein means that there is less than 20% (by weight) of the designated compound or by-product (e.g., a saturated alcohol starting material) present, more preferably, there is less than 10% (by weight) of the designated compound or by-product, more preferably, there is less than 9% (by weight) of the designated compound or by-product, more preferably, there is less than 8% (by weight) of the designated compound or by-product, more preferably, there is less than 7% (by weight) of the designated compound or by-product, more preferably, there is less than 6% (by weight) of the designated compound or by-product, more preferably, there is less than 5% (by weight) of the designated compound or by-product, more preferably, there is less than 4% (by weight) of the designated compound or by-product, more preferably, there is less than 3% (by weight) of the designated compound or by-product, more preferably, there is less than 2% (by weight) of the designated compound or by-product, and most preferably, there is less than 1% (by weight) of the designated compound or by-product.

The term “substituents” refers to a group “attached” to a alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, e.g., C1, C2, C3, C4, C5, C6, C7, C8, C9, C10, C11, C12 straight or branched chain alkyl, cycloalkyl, haloalkyl, e.g., perfluoroalkyl such as CF₃, aryl, heteroaryl, aralkyl, heteroaralkyl, heterocyclyl, alkenyl, alkynyl, cycloalkenyl, heterocycloalkenyl, alkoxy, haloalkoxy, e.g., perfluoroalkoxy such as OCF₃, halo, hydroxy, carboxy, carboxylate, cyano, nitro, amino, alkyl amino, SO₃H, sulfate, phosphate, methylenedioxy e.g., —O—CH₂—O—, ethylenedioxy, oxo, thioxo, e.g., C═S, imino, e.g., alkyl, aryl, aralkyl, S(O)ₙalkyl, S(O)ₙ aryl, S(O)ₙ heteroaryl, S(O)ₙ heterocyclyl, i.e., wherein is an integer between 0 and 2, amine, e.g., mono-, di-, alkyl, cycloalkyl, aralkyl, heteroaralkyl, aryl, heteroaryl, and combinations thereof, ester, e.g., alkyl, aralkyl, heteroaralkyl, aryl, heteroaryl, amide, e.g., mono-, di-, alkyl, aralkyl, heteroaralkyl, aryl, heteroaryl, and combinations thereof, sulfonamide, e.g., mono-, di-, alkyl, aralkyl, heteroaralkyl, and combinations thereof. In one aspect, the substituents on a group are independently any one single, or any subset of the aforementioned substituents. In another aspect, a substituent may itself be substituted with any one of the above substituents.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human” and “subject” are used interchangeably herein.

### Compositions

The present invention relates to a composition such as an anhydrous gel, and its use for example as a cosmetic base, comprising a silicone-based excipient, e.g., a silicone elastomer, and one or more oils, e.g., one or more natural oils such as sunflower seed oil, peppermint oil, eucalyptus oil, and methods of administering the composition to a subject.

It has now been discovered that, by the judicious combination of a silicone elastomer and one or more natural oils in a weight ratio of silicone elastomer to oil(s) of about 1.5 to 0.5, e.g., 1.5 to 0.5, in an anhydrous composition based on volatile aliphatic hydrocarbon(s), liquid fatty esters, lactate esters, particular essential oils and silicone fluids, it is possible to realize clear, gel systems with enhanced aesthetics that impart a luxurious, silky, non-greasy skin feel. Moreover these anhydrous compositions are compatible with a wide range of excipients and other organic ingredients, and perform as quick-absorbing delivery systems for lipophilic actives. In addition, these compositions are water repellant, skin protectant, and amenable to cold processing.

Silicone elastomers are used in the cosmetic industry as thickening agents for both water-based and anhydrous formulations and to enhance the aesthetics of those formulations and provide a novel form of delivery for other formulation components. The elastomers are, in general, compatible with a wide range of organic compounds, including natural oils and silicone fluids. Natural oils are used in cosmetic formulations to provide emolliency and to help maintain the skin barrier and decrease transepidermal water loss (TEWL). For example, sunflower oil, which is rich in linoleic acid, may be particularly beneficial in these respects. Unavoidable consequences of an oil-rich formulation can be an undesirable greasy feel, and long-term residence on the skin. It has been found that the greasy feel of an oil-rich formulation can be mitigated by admixture with silicone elastomer and silicone fluids.

The anhydrous compositions of the instant invention can be rich in emollients like liquid fatty esters and triglycerides and contain a variety of other functional excipients like lactate esters as penetration enhancers; aliphatic hydrocarbons as diluents and to enhance spreadability as well as to help prevent TEWL; silicone fluids to decrease tackiness and enhance overall aesthetics of formulations and for recognized skin protectant effects; and silica silylate for high efficiency viscosity enhancement and fragrance retention. In one embodiment, the compositions comprise cannabis, a product derived from cannabis, e.g., hemp oil, hemp seed oil, or cannabis oil, or a cannabinoid.

The compositions described herein can be formulated for topical or transdermal administration onto a subject. In an embodiment, the subject is a mammal. In an embodiment, the mammal is a human. In an embodiment, a composition described herein is topically administered to a keratinous substrate on the subject, e.g., the skin of a human.

In one aspect, described herein is a composition comprising a silicone-based excipient, a carrier oil, an emollient, and a viscosity enhancement agent, wherein the composition does not comprise a healthcare active. In some embodiments, the viscosity enhancement agent is silica siliylate. In some embodiments, the silicone-based excipient is present in the composition in at least about 80% by weight. In some embodiments, the a silicone-based excipient is a silicone elastomer. In some embodiments, the silicone elastomer is provided in a carrier fluid. In some embodiments, the silicone elastomer is dimethicone/bis-isobutyl polypropylene glycol crosspolymer, e.g., dimethicone/bis-isobutyl PPG-20 crosspolymer. In some embodiments, the carrier fluid is isododecane. In some embodiments, the weight ratio of carrier fluid to silicone elastomer is about 85:15. In some embodiments, the carrier oil is a natural oil. In some embodiments, the carrier oil is selected from the group consisting of sunflower seed oil, coconut oil, grape seed oil, olive oil, jojoba, and combinations thereof. In some embodiments, the carrier oil is sunflower seed oil. In some embodiments, the carrier oil is present in the composition in at least about 5% by weight, e.g., about 5%, about 10%, about 20%, or about 30% by weight. In some embodiments, the weight ratio of silicone elastomer to carrier oil is from about to about 1:0.2 to about 2:1. In some embodiments, the weight ratio of silicone elastomer to carrier oil is about 1.5:0.5. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition further comprises a silicone fluid. In some embodiments, the silicone fluid is a trimethicone. In some embodiments, the trimethicone is phenyl trimethicone. In some embodiments, the composition is an anhydrous gel. In some embodiments, the composition comprises less than 0.5% of water by weight. In some embodiments, the composition further comprises an additional essential oil. In some embodiments, the essential oil is selected from the group consisting of birch oil, lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1% by weight, e.g., 1%, 3%, 4%, 5%, 10%, 20%, or 30% by weight. In some embodiments, the composition further comprises a terpenoid, e.g., camphor. In some embodiments, the composition further comprises a wax, e.g., beeswax. In some embodiments, the composition further comprises cannabis or a product derived from cannabis. e.g., a product derived from cannabis such as hemp oil, hemp seed oil, or cannabis oil. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In one aspect, described herein is a composition comprising a silicone-based excipient, a carrier oil, an emollient, and a viscosity enhancement agent, wherein the composition does not comprise lidocaine, fluocinonide, or tacrolimus. In some embodiments, the viscosity enhancement agent is silica siliylate. In some embodiments, the silicone-based excipient is present in the composition in at least about 80% by weight. In some embodiments, the a silicone-based excipient is a silicone elastomer. In some embodiments, the silicone elastomer is provided in a carrier fluid. In some embodiments, the silicone elastomer is dimethicone/bis-isobutyl polypropylene glycol crosspolymer, e.g., dimethicone/bis-isobutyl PPG-20 crosspolymer. In some embodiments, the carrier fluid is isododecane. In some embodiments, the weight ratio of carrier fluid to silicone elastomer is about 85:15. In some embodiments, the carrier oil is a natural oil. In some embodiments, the carrier oil is selected from the group consisting of sunflower seed oil, coconut oil, grape seed oil, olive oil, jojoba, and combinations thereof. In some embodiments, the carrier oil is sunflower seed oil. In some embodiments, the carrier oil is present in the composition in at least about 5% by weight, e.g., about 5%, about 10%, about 20%, or about 30% by weight. In some embodiments, the weight ratio of silicone elastomer to carrier oil is from about to about 1:0.2 to about 2:1. In some embodiments, the weight ratio of silicone elastomer to carrier oil is about 1.5:0.5. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition further comprises a silicone fluid. In some embodiments, the silicone fluid is a trimethicone. In some embodiments, the trimethicone is phenyl trimethicone. In some embodiments, the composition is an anhydrous gel. In some embodiments, the composition comprises less than 0.5% of water by weight. In some embodiments, the composition further comprises an additional essential oil. In some embodiments, the essential oil is selected from the group consisting of birch oil, lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1%, e.g., 1%, 3%, 4%, 5%, 10%, 20%, or 30% by weight. In some embodiments, the composition further comprises cannabis or a product derived from cannabis. In some embodiments, the product derived from cannabis is selected from the group consisting of cannabis oil, hemp oil, and hemp seed oil. In some embodiments, the composition further comprises a cannabinoid, e.g., a cannabinoid described herein. In some embodiments, the cannabinoid is derived from a naturally occurring source. In some embodiments, the cannabinoid is a synthetic cannabinoid. In some embodiments, the composition further comprises a terpenoid, e.g., camphor. In some embodiments, the composition further comprises a wax, e.g., beeswax. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In another aspect, described herein is a composition formulated for topical or transdermal delivery, the composition comprising a cannabinoid, a carrier oil, a silicone elastomer, and a viscosity enhancement agent, e.g., silica silylate. In some embodiments, the composition is anhydrous, e.g., comprising less than 0.5% of water by weight. In some embodiments, the composition is an anhydrous gel. In some embodiments, the silicone-based excipient is present in the composition in at least about 80% by weight. In some embodiments, the silicone elastomer is dimethicone/bis-isobutyl polypropylene glycol crosspolymer, e.g., dimethicone/bis-isobutyl PPG-20 crosspolymer. In some embodiments, the silicone elastomer is provided in a carrier fluid. In some embodiments, the carrier fluid is isododecane. In some embodiments, the weight ratio of carrier fluid to silicone elastomer is about 85:15. In some embodiments, the composition further comprises an oil, e.g., a natural oil, e.g., sunflower seed oil, peppermint oil, eucalyptus oil, cannabis oil, or a combination thereof. In some embodiments, the weight ratio of silicone elastomer to oil is from about to about 1:0.2 to about 2:1. In some embodiments, the weight ratio of silicone elastomer to oil is about 1.5:0.5. In some embodiments, the composition comprises an ester. In some embodiments, the ester is a lactate ester. In some embodiments, the lactate ester is lauryl lactate. In some embodiments, the ester is present in the composition in an amount of less than about 0.5% by weight. In some embodiments, the ester is present in the composition in an amount of from about 0.5% to about 5% by weight. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition is substantially free of an antimicrobial. In some embodiments, the composition further comprises a silicone fluid. In some embodiments, the silicone fluid comprises a trimethicone. In some embodiments, the trimethicone is phenyl trimethicone. In some embodiments, the cannabinoid is derived from a naturally occurring source, e.g., a natural oil such as cannabis oil, hemp oil, and hemp seed oil. In some embodiments, the cannabinoid is a synthetic cannabinoid. In some embodiments, the cannabinoid is a cannabinoid described herein. In some embodiments, the composition comprises a plurality of cannabinoids. In some embodiments, the plurality of cannabinoids comprises cannabidiol and tetrahydrocannabinol. In some embodiments, the cannabidiol comprises a synthetic cannabidiol. In some embodiments, the cannabinoid is a cannabinoid described herein. In some embodiments, the composition does not comprise diethylene glycol monoethyl ether. In some embodiments, the composition further comprises an additional essential oil. In some embodiments, the essential oil is selected from the group consisting of birch oil, lavender oil, eucalyptus oil, peppermint oil, a high terpene oil, and combinations thereof. In some embodiments, the essential oil is selected from the group consisting of lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1% by weight, e.g., 1%, 3%, 4%, 5%, 10%, 20%, or 30% by weight. In some embodiments, the composition is substantially free of an antimicrobial. In some embodiments, the composition does not comprise diethylene glycol monoethyl ether. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In another aspect, described herein is a composition, the composition comprising a carrier oil, an essential oil, an emollient, a silicone elastomer, and a cannabinoid. In some embodiments, the composition further comprises a viscosity enhancement agent, e.g., silica silylate. In some embodiments, the composition is anhydrous, e.g., comprising less than 0.5% of water by weight. In some embodiments, the composition is an anhydrous gel. In some embodiments, the silicone-based excipient is present in the composition in at least about 80% by weight. In some embodiments, the silicone elastomer is dimethicone/bis-isobutyl polypropylene glycol crosspolymer, e.g., dimethicone/bis-isobutyl PPG-20 crosspolymer. In some embodiments, the silicone elastomer is provided in a carrier fluid. In some embodiments, the carrier fluid is isododecane. In some embodiments, the weight ratio of carrier fluid to silicone elastomer is about 85:15. In some embodiments, the composition further comprises an oil, e.g., a natural oil, e.g., sunflower seed oil, peppermint oil, eucalyptus oil, cannabis oil, or a combination thereof. In some embodiments, the weight ratio of silicone elastomer to oil is from about to about 1:0.2 to about 2:1. In some embodiments, the weight ratio of silicone elastomer to oil is about 1.5:0.5. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition is substantially free of an antimicrobial. In some embodiments, the composition further comprises a silicone fluid. In some embodiments, the silicone fluid comprises a trimethicone. In some embodiments, the trimethicone is phenyl trimethicone. In some embodiments, the composition does not comprise diethylene glycol monoethyl ether. In some embodiments, the essential oil is selected from the group consisting of birch oil, lavender oil, eucalyptus oil, peppermint oil, a high terpene oil, and combinations thereof. In some embodiments, the essential oil is selected from the group consisting of lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1% by weight, e.g., 1%, 3%, 4%, 5%, 10%, 20%, or 30% by weight. In some embodiments, the composition further comprises cannabis or a product derived from cannabis. In some embodiments, the cannabinoid is a cannabinoid described herein. In some embodiments, the composition comprises a plurality of cannabinoids, e.g., a plurality of cannabinoids described herein. In some embodiments, the cannabinoid is derived from a naturally occurring source, e.g., cannabis oil, hemp oil, and hemp seed oil. In some embodiments, the cannabinoid is a synthetic cannabinoid. In some embodiments, the composition is substantially free of an antimicrobial. In some embodiments, the composition does not comprise diethylene glycol monoethyl ether. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In an aspect, described herein is a method of adding an additional component to a composition, e.g., a composition formulated for topical or transdermal administration, comprising a silicone-based excipient, a carrier oil, an emollient, and a viscosity enhancement agent. In some embodiments, the additional component is an essential oil. In some embodiments, the essential oil is selected from the group consisting of birch oil, lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is added to the composition in an amount that is at least about 1%, e.g., 1%, 3%, 4%, 5%, 10%, 20%, or 30%, of the total weight of the composition. In some embodiments, the additional component comprises cannabis or a product derived from cannabis. In some embodiments, the product derived from cannabis is hemp oil, hemp seed oil, or cannabis oil. In some embodiments, the additional component comprises a cannabinoid, e.g., a cannabinoid described herein. In some embodiments, the additional component comprises a plurality of cannabinoids, e.g., a plurality of cannabinoids described herein. In some embodiments, the plurality of cannabinoids comprises cannabidiol and tetrahydrocannabinol. In some embodiments, the cannabidiol comprises a synthetic cannabidiol. In some embodiments, the cannabidiol is derived from a natural oil, e.g., cannabis oil, hemp oil, or hemp seed oil. In some embodiments, the additional component comprises an essential oil, e.g., an essential oil described herein, and a cannabinoid, e.g., a cannabinoid described herein. In some embodiments, the additional component comprises an essential oil, e.g., an essential oil described herein, and cannabis or a product derived from cannabis, e.g., a product derived from cannabis such as hemp oil, hemp seed oil, or cannabis oil. In some embodiments the cannabinoid is derived from a naturally-occurring source, e.g., a natural oil such as hemp oil, hemp seed oil, or cannabis oil. In some embodiments, the essential oil is present in the composition in at least about 1% by weight, e.g., 1%, 3%, 4%, 5%, 10%, 20%, or 30% by weight. In some embodiments, the cannabinoid is present in the composition in at least about 1% by weight, e.g., 1%, 3%, 4%, 5%, 10%, 20%, or 30% by weight. In some embodiments, the additional component comprises a terpenoid, e.g., camphor. In some embodiments, the composition is administered on a keratinous surface of the subject. In some embodiments, the keratinous surface is the skin of the subject. In some embodiments, the subject is a mammal. In some embodiments, the mammal is a human.

The compositions provided herein can also be administered by a transdermal device. Accordingly, transdermal administration can be accomplished using a patch either of the reservoir or porous membrane type, or of a solid matrix variety.

### Carrier Oils

The compositions of the present invention can comprise a carrier oil, e.g., a natural oils, e.g., oils derived from plants. Non-limiting examples of natural oils include sunflower seed oil, coconut oil, grape seed oil, hemp seed oil, olive oil, hemp oil, and jojoba. The composition can comprise a combination of carrier oils, including but not limited to any combination of sunflower seed oil, coconut oil, grape seed oil, hemp seed oil, olive oil, hemp oil, and jojoba.

In some embodiments, the carrier oil is present in the composition from about 1% by weight to about 90% by weight. In some embodiments, the carrier oil is present in the composition from about 1% by weight to about 50% by weight. In some embodiments, the carrier oil is present in the composition from about 1% by weight to about 40% by weight. In some embodiments, the carrier oil is present in the composition from about 1% by weight to about 30% by weight. In some embodiments, the carrier oil is present in the composition from about 1% by weight to about 20% by weight. In some embodiments, the carrier oil is present in the composition from about 1% by weight to about 10% by weight. In some embodiments, the carrier oil is present in the composition from about 1% by weight to about 5% by weight. In some embodiments, the carrier oil is present in the composition in about 30% by weight, e.g., 30% by weight. In some embodiments, the carrier oil is present in the composition in about 20% by weight, e.g., 20% by weight. In some embodiments, the carrier oil is present in the composition in about 15% by weight, e.g., 15% by weight. In some embodiments, the carrier oil is present in the composition in about 14.5% by weight, e.g., 14.5% by weight. In some embodiments, the carrier oil is present in the composition in about 14% by weight, e.g., 14% by weight. In some embodiments, the carrier oil is present in the composition in about 13.7% by weight, e.g., 13.7% by weight. In some embodiments, the carrier oil is present in the composition in about 10% by weight, e.g., 10% by weight. In some embodiments, the carrier oil is present in the composition in about 7.5% by weight, e.g., 7.5% by weight or 7.6% by weight. In some embodiments, the carrier oil is present in the composition in about 5% by weight, e.g., 5% by weight. In some embodiments, the carrier oil is present in the composition in about 2.5% by weight, e.g., 2.5% by weight. In some embodiments, the carrier oil is present in the composition in about 1% by weight, e.g., 1% by weight.

### Silicone-Based Excipient

The silicone-based excipient may be any silicone-containing polymer material, including a silicone elastomer blend, a silicone organic elastomer blend, a silicone resin, a silicone elastomer, a pressure sensitive adhesive, a silicone gum, a silicone wax, an elastomer base sealant, adhesive or any combination thereof. The silicone-based excipient may be a dimethicone crosspolymer, a dimethicone/bis-isobutyl propylene glycol crosspolymer, a polyethylene glycol-12 dimethicone/bis-isobutyl propylene glycol-20 crosspolymer, or any combination thereof.

Silicones are a class of compounds based on polydialkylsiloxanes. Silicones have been used extensively to enhance aesthetics of personal care formulations by providing a unique sensory profile upon application. Silicone elastomer gels are generally obtained by a crosslinking hydrosilylation reaction of a SiH polysiloxane with another polysiloxane containing an unsaturated hydrocarbon substituent, such as a vinyl functional polysiloxane, or by crosslinking a SiH polysiloxane with a hydrocarbon diene. The silicone elastomers may be formed in the presence of a carrier fluid, such as a volatile silicone, resulting in a gelled formulation. The silicone-based excipient may be a pressure sensitive adhesive (PSA). The PSA may be the reaction product of a hydroxyl end-blocked polydimethylsiloxane polymer and a hydroxy functional silicate resin. The polymer and resin react in a condensation reaction to form the PSA. The advantage of using the PSA as the silicone component is the substantivity that the PSA provides.

For purposes of the present disclosure, the terms “silicone rubber” and “silicone elastomer” are synonymous, at least to the extent that both silicone components are capable of elongation and recovery. The silicone elastomers may be contained in a carrier fluid such as cyclopentasiloxane, isododecane, isodecylneopentanoate, caprylyl methicone, or other suitable carrier fluids. Silicone rubbers and silicone elastomers are generally crosslinked or reacted silicone polymers. In contrast, silicone gums are capable of being stretched, but they do not generally snap back. Silicone gums are the high molecular weight, generally linear, polydiorganosiloxanes that can be converted from their highly viscous plastic state into a predominately elastic state by crosslinking. Silicone gums are often used as one of the main components in the preparation of silicone rubbers and silicone elastomers.

The compositions described herein can comprise one or more silicone elastomers. In some embodiments, the silicone elastomer is a dimethicone/bis-isobutyl polypropylene glycol crosspolymer, e.g., dimethicone/bis-isobutyl PPG-20 crosspolymer. In some embodiments, the silicone elastomer is provided in a carrier fluid. In some embodiments, the carrier fluid is isododecane. In some embodiments, the weight ratio of carrier solvent to silicone elastomer is about 85:15.

The silicone resins may include MQ resins. The acronym MQ as it relates to silicone resins is derived from the symbols M, D, T, and Q each of which represent a functionality of different types of structural units which may be present in silicone resins containing siloxane units joined by Si—O—Si bonds. Monofunctional (M) unit represents (CH₃)₃ SiO_{1/2}. Difunctional (D) unit represents (CH₃)₂SiO_{2/2}. Trifunctional (T) unit represents CH₃SiO_{3/2} and results in the formation of branched linear siloxanes. Tetrafunctional (Q) unit represents SiO_{4/2} which results in the formation of crosslinked and resinous silicone compositions. Hence, MQ is used when the siloxane contains all monofunctional M and tetrafunctional Q units, or at least a high percentage of M and Q units such as to render the silicone resinous.

Silicone resins may include non-linear siloxane resins having a glass transition temperature (T_{g}) above about 0° C. Glass transition temperature is the temperature at which an amorphous material such as a higher silicone polymer changes from a brittle vitreous state to a plastic state. The silicone resin generally has the formula R′ₐSiO_{(4-a)/2} wherein R′ is a monovalent hydrocarbon group with 1-6 carbon atoms or a functionally substituted hydrocarbon group with 1-6 carbon atoms, and a has an average value of 1-1.8. The silicone resin will preferably include monofunctional (M) units R″₃SiO_{1/2} and tetrafunctional (Q) units SiO_{4/2}, in which R″ is the monovalent hydrocarbon group having 1-6 carbon atoms, most preferably the methyl group. The number ratio of M groups to Q groups may be in the range of 0.5:1 to 1.2:1, so as to provide an equivalent wherein a in the formula R′ₐSiO_{(4-a)/2} has an average value of 1.0-1.63. The number ratio of M groups to Q groups may also be between about 0.6:1 to about 0.9:1. Silicone MQ resins in which the number of Q units per molecule is higher than 1 or higher than 5 may also be used.

The silicone resin may also contain between about 1 to about 5% by weight of silicon-bonded hydroxyl radicals such as a dimethylhydroxysiloxy unit (HO)(CH₃)₂SiO_{1/2}. If desired, the silicone resin may contain minor amounts of difunctional (D) units and/or trifunctional (T) units. Silicone resins having a viscosity of at least 100,000,000 (100 million) centistoke (mmf²/s) and a softening temperature of less than about 200° C. may also be used. The silicone resin may include (i) silicone resins of the type MₓQ_{y} where x and y have values such that the silicone resin contains at least more than 5 Q units per molecule; (ii) silicone resins of the type MₓT_{y} where x and y have values such that the silicone resin contains at least more than 5 T units per molecule; and (iii) silicone resins of the type MₓD_{y}TₚQ_{q} where x, y, p, and q have values such that the sum of Q and T units is at least more than 5 units per molecule, and the number of D units varies from 0-100.

The compositions described herein can comprise one or more silicone-based excipients. In some embodiments, the silicone-based excipient, e.g., a silicone elastomer provided in a carrier fluid such as isododecane, is present in the composition in least about 50% by weight, in at least about 55% by weight, in least about 60% by weight, in at least about 65% by weight, in least about 70% by weight, in at least about 75% by weight, in at least about 80% by weight, in at least about 85% by weight, in at least about 90% by weight, in at least about 95% by weight, or in at least about 99% by weight. In some embodiments, the silicone-based excipient, e.g., a silicone elastomer provided in a carrier fluid such as isododecane, is present in the composition in least about 55% by weight, e.g., 55% by weight. In some embodiments, the silicone-based excipient, e.g., a silicone elastomer provided in a carrier fluid such as isododecane, is present in the composition in least about 55% by weight, e.g., 55% by weight. In some embodiments, the silicone-based excipient, e.g., a silicone elastomer provided in a carrier fluid such as isododecane, is present in the composition in least about 65% by weight, e.g., 65% by weight. In some embodiments, the silicone-based excipient, e.g., a silicone elastomer provided in a carrier fluid such as isododecane, is present in the composition in least about 67.4% by weight, e.g., 67.4% by weight.

### Volatile Solvent

The formulation according to the present disclosure can include a volatile solvent. The silicone-based excipient may be contained in volatile solvent (or carrier fluid) to provide the present topical formulations. Typically, the volatile solvent is the solvent used for conducting the hydrosilylation reaction to form the silicone-based excipient. Suitable volatile solvents include volatile solvents, organic liquids (oils and solvents), silicones and mixtures thereof.

Solvents may include volatile liquids such as alcohols (e.g., methyl, ethyl, isopropyl alcohols and methylene chloride); ketones (e.g., acetone); aromatic hydrocarbons such as benzene derivatives (e.g., xylenes and toluenes); lower molecular weight alkanes and cycloalkanes (e.g., hexanes, heptanes and cyclohexanes); and alkanoic acid esters (e.g., ethyl acetate, n-propyl acetate, isobutyl acetate, n-butyl acetate, isobutyl isobutyrate, hexyl acetate, 2-ethylhexyl acetate or butyl acetate); and combinations and mixtures thereof.

Typically, the volatile solvent is an organic liquid. Organic liquids include oils and solvents. The organic liquids are exemplified by, but not limited to, aromatic hydrocarbons, aliphatic hydrocarbons, alcohols, aldehydes, ketones, amines, esters, ethers, glycols, glycol ethers, alkyl halides and aromatic halides. Hydrocarbons include isododecane, isohexadecane, Isopar L (C11-C13), Isopar H (C11-C12), hydrogentated polydecene. Ethers and esters include, isodecyl neopentanoate, neopentylglycol heptanoate, glycol distearate, dicaprylyl carbonate, diethylhexyl carbonate, propylene glycol n-butyl ether, ethyl-3 ethoxypropionate, propylene glycol methyl ether acetate, tridecyl neopentanoate, propylene glycol methylether acetate (PGMEA), propylene glycol methylether (PGME), octyldodecyl neopentanoate, diisobutyl adipate, diisopropyl adipate, propylene glycol dicaprylate/dicaprate, and octyl palmitate. Additional volatile solvents suitable as a standalone compound or as an ingredient to the carrier fluid include fats, oils, fatty acids, and fatty alcohols.

The volatile solvent may also be a low viscosity organopolysiloxane or a volatile methyl siloxane or a volatile ethyl siloxane or a volatile methyl ethyl siloxane having a viscosity at 25° C. in the range of about 1 to about 1,000 mm²/sec, exemplified by hexamethylcyclotrisiloxane, octamethyleyelotetrasiloxane, decamethylcyclopentasiloxane, dodecamethylcyclohexasiloxane, octamethyltrisiloxane, decamethyltetrasiloxane, dodecamethylpentasiloxane, tetradecamethylhexasiloxane, hexadeamethylheptasiloxane, heptamethyl-3-{(trimethylsilyl)oxy)}trisiloxane, hexamethyl-3,3,bis{(trimethlylsilyl)oxy}trisiloxane pentamethyl{(trimethylsilyl)oxy}cyclotrisiloxane as well as polydimethylsiloxanes, polyethylsiloxanes, polymethylethylsiloxanes, polymethylphenylsiloxanes, polydiphenylsiloxanes.

### Emollients

An emollient is a hydrophobic material that provides softness, lubricity and smoothness to the skin and often forms a thin occlusive film that increases hydration by reducing transepidermal water loss (TEWL). Exemplary hydrophobic emollients include, but are not limited to, short chain (i.e., C1-C6) alkyl or (C6-C12) aryl esters of long (i.e., C8-C36) straight or branched chain alkyl or alkenyl alcohols or acids and polyethoxylated derivatives of the alcohols; short chain (i.e., C1-C6) alkyl or (C6-C12) aryl esters of (C4-C12) diacids or (C4-C12) diols optionally substituted in available positions by —OH; (C2-C18) alkyl or (C6-C12) aryl esters of glycerol, pentaerythritol, ethylene glycol, propylene glycol, as well as polyethoxylated derivatives of these; (C12-C22) alkyl esters or (C12-C22) ethers of polypropylene glycol; (C12-C22) alkyl esters or (C12-C22) ethers of polypropylene glycol/polyethylene glycol copolymer; and polyether polysiloxane copolymers. Additional examples of hydrophobic components include cyclic dimethicones, including volatile cyclic silicones such as D4 and D5, polydialkylsiloxanes, polyaryl/alkylsiloxanes, silicone copolyols, cocoa butter, beeswax, jojoba oil, lanolin and derivatives, long chain (i.e., C8-C36) alkyl and alkenyl esters of long (i.e., C8-C18) straight or branched chain alkyl or alkenyl alcohols or acids, long chain (i.e., C8-C36) alkyl and alkenyl amides of long straight or branched chain (i.e., C8-C36) alkyl or alkenyl amines or acids; hydrocarbons including straight and branched chain alkanes and alkenes such as isoparafins (e.g., isooctane, isododecane, isooctadecane, etc.), squalene, and mineral oil, polysiloxane polyalkylene copolymers, dialkoxy dimethyl polysiloxanes; (C12-C22) alkyl and (C12-C22) alkenyl alcohols, and petroleum derived alkanes such as isoparafins, petrolatum, petrolatum USP, as well as refined natural oils (especially NF or USP grades) such as olive oil NF, cotton seed oil, castor oil, peanut oil, corn oil, seasame oil, safflower oil, soybean oil, sunflower oil and the like, and blends thereof. In certain preferred embodiments, the hydrophobic components useful in the compositions of the present invention include those selected from the group consisting of petrolatum USP and short chain (i.e., C1-C6) alkyl or (C6-C12) aryl esters of long (i.e., C8-C36) straight or branched chain alkyl or alkenyl alcohols or acids and polyethoxylated derivatives of the alcohols; short chain (i.e., C1-C6) alkyl or (C6-C12) aryl esters of (C4-C12) diacids or (C4-C12) diols optionally substituted in available positions by —OH (such as diisopropyladipate, diisopropylsebacate); (C1-C9) alkyl or (C6-C12) aryl esters of glycerol, pentaerythritol, ethylene glycol, propylene glycol (such as glyceryl tricaprylate/caprate); and mixtures thereof. In some embodiments, the emollient is the ester of a fatty acid, e.g., a triglyceride. In some embodiments, the triglyceride is capric triglyceride or caprylic triglyceride. In some embodiments, the emollient is an ester described herein.

In some embodiments, the emollient is present in the composition from about 0% by weight to about 25% by weight. In some embodiments, the emollient is present in the composition from about 1% by weight to about 10% by weight. In some embodiments, the emollient is present in the composition from about 1% by weight to about 5% by weight. In some embodiments, the emollient is present in the composition in about 10% by weight, e.g., 10% by weight. In some embodiments, the emollient is present in the composition in about 7.5% by weight, e.g., 7.5% by weight or 7.6% by weight. In some embodiments, the emollient is present in the composition in about 5% by weight, e.g., 5% by weight. In some embodiments, the emollient is present in the composition in about 2.5% by weight, e.g., 2.5% by weight. In some embodiments, the emollient is present in the composition in about 1% by weight, e.g., 1% by weight.

### Esters

Compositions of the present invention can comprise one or more esters. The ester can be a lactate ester, for example lauryl lactate, myristyl lactate, or cetyl lactate.

The compositions described herein can comprise an α-hydroxy carboxylic acid ester and a second ester, wherein the α-hydroxy carboxylic acid ester and the second ester are not the same; and wherein less than 10% (e.g., by weight or volume) of the composition of esters comprises fatty alcohols and/or an alkyl lactyl lactate and/or other related substances. In some embodiments, at least 90% (e.g., by weight or volume) of the composition is comprised of esters.

In certain embodiments, the second ester is a compound of formula (I):

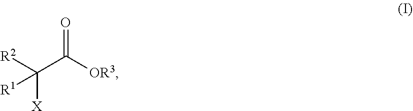

wherein each R¹ is independently selected from the group consisting of: H, C₁-C₂₀ straight chained or branched alkyl, C₃-C₁₀ cycloalkyl, substituted C₁-C₂₀ alkyl, arylalkyl, aryl, substituted aryl and heteroaryl; each R² is independently selected from the group consisting of: H and C₁-C₂₀ alkyl; R³ is a C₁₂-C₄₀ alkyl; and X is NR¹R¹ or OR¹;

In some embodiments, the α-hydroxy carboxylic acid ester is a compound of formula (II):

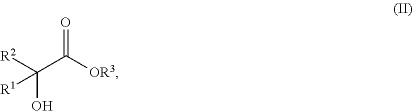

wherein R¹, R² and R³ are as defined in formula (I).

In some embodiments, the second ester is a compound of formula (II):

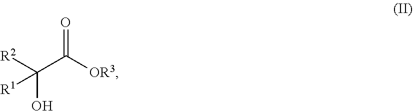

wherein R¹, R² and R³ are as defined in formula (I).

In some embodiments, the α-hydroxy carboxylic acid ester and the second esters are compounds of formula (II):

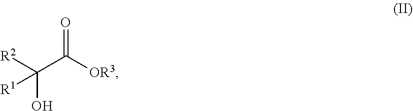

wherein R¹, R² and R³ are as described in formula (I).

In certain embodiments, the composition further comprises a third ester. In some embodiments, the composition comprises lauryl lactate, myristyl lactate, and cetyl lactate. In some embodiments, the composition further comprises a fourth ester. In some embodiments, the composition further comprises a fifth ester. In some embodiments, the composition comprises more than five or more esters.

In certain embodiments, the α-hydroxy carboxylic acid ester, the second ester and the third ester are not the same. In some embodiments, the α-hydroxy carboxylic acid ester, the second ester, the third ester and the fourth ester are not the same. In some embodiments, the second ester, the third ester, the fourth ester and the fifth are not the same.

In certain embodiments, the α-hydroxy carboxylic acid ester and the second ester used in the composition are selected from the group consisting of lauryl lactate, lauryl mandelate, myristyl lactate, cetyl lactate, stearyl lactate, ethyl hexyl lactate and dimethyl octyl lactate. In some embodiments, the α-hydroxy carboxylic acid ester, the second ester and the third ester used in the composition are selected from the group consisting of lauryl lactate, myristyl lactate, cetyl lactate, stearyl lactate, ethyl hexyl lactate and dimethyl octyl lactate. In some embodiments, the α-hydroxy carboxylic acid ester, the second ester, the third ester and the fourth ester used in the composition are selected from the group consisting of lauryl lactate, myristyl lactate, cetyl lactate, stearyl lactate, ethyl hexyl lactate and dimethyl octyl lactate. In some embodiments, the α-hydroxy carboxylic acid ester, the second ester, the third ester, the fourth ester and the fifth ester used in the composition are selected from the group consisting of lauryl lactate, myristyl lactate, cetyl lactate, stearyl lactate, ethyl hexyl lactate and dimethyl octyl lactate. In some embodiments, the esters are chosen in ratios so as to provide a preselected delivery of a drug through the skin or mucousal membrane.

In certain embodiments, the composition comprises less than 5% (e.g., by weight or volume) fatty alcohols and/or alkyl lactyllactate and/or other related substances. In certain embodiments, the composition comprises less than 3% (e.g., by weight or volume) fatty alcohols and/or alkyl lactyllactate. In certain embodiments, the composition comprises less than 1% (e.g., by weight or volume) fatty alcohols and/or alkyl lactyllactate. In some embodiments, the composition is substantially free of fatty alcohols and/or alkyl lactyllactate.

In certain embodiments, the α-hydroxy carboxylic acid ester is present in a racemic mixture (e.g., less than 10% enantiomeric excess of either the R or S stereoisomer). In some embodiments, the α-hydroxy carboxylic acid ester is present in an enantiomeric excess of the R stereoisomer (e.g., 10%, 50%, 75%, 85%, 90%, 95%, 97% or greater). In some embodiments, the α-hydroxy carboxylic acid ester is present in an enantiomeric excess of the S stereoisomer (e.g., 10%, 50%, 75%, 85%, 90%, 95%, 97% or greater).

In certain embodiments, the second ester is present in a racemic mixture (e.g., less than 10% enantiomeric excess of either the R or S stereoisomer). In some embodiments, the second ester is present in an enantiomeric excess of the R stereoisomer (e.g., 10%, 50%, 75%, 85%, 90%, 95%, 97% or greater). In some embodiments, the second ester is present in an enantiomeric excess of the S stereoisomer (e.g., 10%, 50%, 75%, 85%, 90%, 95%, 97% or greater).

In certain embodiments, the third ester is present in a racemic mixture (e.g., less than 10% enantiomeric excess of either the R or S stereoisomer). In some embodiments, the third ester is present in an enantiomeric excess of the R stereoisomer (e.g., 10%, 50%, 75%, 85%, 90%, 95%, 97% or greater). In some embodiments, the third ester is present in an enantiomeric excess of the S stereoisomer (e.g., 10%, 50%, 75%, 85%, 90%, 95%, 97% or greater).

In certain embodiments, the fourth ester is present in a racemic mixture (e.g., less than 10% enantiomeric excess of either the R or S stereoisomer). In some embodiments, the fourth ester is present in an enantiomeric excess of the R stereoisomer (e.g., 10%, 50%, 75%, 85%, 90%, 95%, 97% or greater). In some embodiments, the fourth ester is present in an enantiomeric excess of the S stereoisomer (e.g., 10%, 50%, 75%, 85%, 90%, 95%, 97% or greater).

In certain embodiments, the fifth ester is present in a racemic mixture (e.g., less than 10% enantiomeric excess of either the R or S stereoisomer). In some embodiments, the fifth ester is present in an enantiomeric excess of the R stereoisomer (e.g., 10%, 50%, 75%, 85%, 90%, 95%, 97% or greater). In some embodiments, the fifth ester is present in an enantiomeric excess of the S stereoisomer (e.g., 10%, 50%, 75%, 85%, 90%, 95%, 97% or greater).

In certain embodiments, the ester is a compound of formula (I-A):

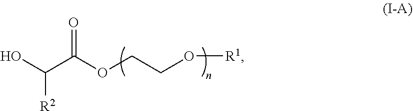

wherein
R¹ is a C₈₋₃₅ alkyl;
n is an integer from 2 to 35; and
R² is hydrogen, alkyl, aryl, heteroaryl, heterocyclyl or aralkyl;
or a pharmaceutically acceptable salt thereof.

In certain embodiments, n is an integer from 2 to 25. In some embodiments, n is an integer from 2 to 15. In some embodiments, n is an integer from 2 to 10. In some embodiments, n is an integer from 4 to 10. In some embodiments, n is 2. In some embodiments, n is 6. In some embodiments, n is 7. In some embodiments, n is 8. In some embodiments, n is 9. In some embodiments, n is 10. In some embodiments, n is 11. In some embodiments, n is 12.

In certain embodiments, R¹ is a C₈₋₂₅ alkyl group. In some embodiments, R¹ is a C₈₋₂₀ alkyl group. In some embodiments, R¹ is a C₁₀₋₁₅ alkyl group. In some embodiments, R¹ is a C₁₂ alkyl group.

In certain embodiments, R² is alkyl (e.g., C₁₋₄ alkyl). In certain embodiments, R² is C₁₋₄ alkyl (e.g., methyl). In certain embodiments, R² is aryl (e.g., phenyl). In some embodiments, R² is aralkyl (e.g., benzyl).

In certain embodiments, the compound of formula (I-A) is represented by the following formula:

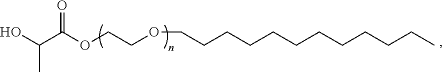

wherein n is an integer from 2 to 35. In some aspects of this embodiment, n is an integer from 2 to 25. In some aspects of this embodiment, n is an integer from 2 to 15. In some aspects of this embodiment, n is an integer from 2 to 10. In some embodiments, n is an integer from 4 to 10. In some embodiments, n is 6. In some embodiments, n is 7. In some embodiments, n is 8. In some embodiments, n is 9. In some embodiments, n is 10. In some embodiments, n is 11. In some embodiments, n is 12.

In some embodiments, the compound of formula (I-A) is represented by the following formula:

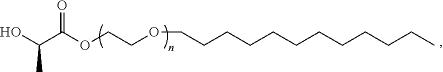

wherein n is an integer from 2 to 35. In some aspects of this embodiment, n is an integer from 2 to 25. In some aspects of this embodiment, n is an integer from 2 to 15. In some aspects of this embodiment, n is an integer from 2 to 10. In some aspects of this embodiment, n is an integer from 4 to 10. In some embodiments, n is 6. In some embodiments, n is 7. In some embodiments, n is 8. In some embodiments, n is 9. In some embodiments, n is 10. In some embodiments, n is 11. In some embodiments, n is 12.

In some embodiments, the compound of formula (I-A) is represented by the following formula:

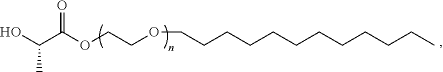

wherein n is an integer from 2 to 35. In some aspects of this embodiment, n is an integer from 2 to 25. In some aspects of this embodiment, n is an integer from 2 to 15. In some aspects of this embodiment, n is an integer from 2 to 10. In some embodiments, n is an integer from 4 to 10. In some embodiments, n is 6. In some embodiments, n is 7. In some embodiments, n is 8. In some embodiments, n is 9. In some embodiments, n is 10. In some embodiments, n is 11. In some embodiments, n is 12.

In certain embodiments, the compound of formula (I-A) may have an average molecular weight ranging from about 300 daltons to about 2000 daltons (e.g., from about 300 daltons to about 1500 daltons, from about 400 daltons to about 1200 daltons, from about 500 daltons to about 1000 daltons, from about 600 daltons to about 850 daltons).

The polymeric portion of a compound of formula (I-A) described herein may have a polymer polydispersity index (PDI) of less than or equal to about 2.5 (e.g., less than or equal to about 2.2, less than or equal to about 2.0, or less than or equal to about 1.5). In some embodiments, a hydrophobic polymer described herein may have a polymer PDI of about 1.0 to about 2.5, about 1.0 to about 2.0, about 1.0 to about 1.7, or from about 1.0 to about 1.6.

In certain embodiments, the compound of formula (I-A) is a racemic mixture (e.g., less than 10% enantiomeric excess of either the R or S stereoisomer). In certain embodiments, the compound of formula (I-A) is at least 10% enantiomeric excess of the R stereoisomer. In certain embodiments, the compound of formula (I-A) is at least 50% enantiomeric excess of the R stereoisomer. In some embodiments, the compound of formula (I-A) is at least 75% enantiomeric excess of the R stereoisomer. In some embodiments, the compound of formula (I-A) is at least 85% enantiomeric excess of the R stereoisomer. In some embodiments, the compound of formula (I-A) is at least 90% enantiomeric excess of the R stereoisomer. In some embodiments, the compound compound of formula (I-A) is at least 95% enantiomeric excess of the R stereoisomer. In some embodiments, the compound of formula (I-A) is least 97% enantiomeric excess of the R stereoisomer. In some embodiments, the compound of formula (I-A) is at least 99% enantiomeric excess of the R stereoisomer.

In certain embodiments, the formula (I-A) is at least 10% enantiomeric excess of the S stereoisomer. In certain embodiments, the compound of formula (I-A) is at least 50% enantiomeric excess of the S stereoisomer. In some embodiments, the compound of formula (I-A) is at least 75% enantiomeric excess of the S stereoisomer. In some embodiments, the compound of formula (I-A) is at least 85% enantiomeric excess of the S stereoisomer. In some embodiments, the compound of formula (I-A) is at least 90% enantiomeric excess of the S stereoisomer. In some embodiments, the compound of formula (I-A) is at least 95% enantiomeric excess of the S stereoisomer. In some embodiments, the compound of formula (I-A) is at least 97% enantiomeric excess of the S stereoisomer. In some embodiments, the compound of formula (I-A) is at least 99% enantiomeric excess of the S stereoisomer.

### Essential Oils

The compositions of the present invention can comprise one or more essential oils. Non-limiting examples of essential oils include birch oil, lavender oil, eucalyptus oil, peppermint oil, and a high terpene oil. In a preferred embodiment, a composition described herein comprises birch oil.

In some embodiments, the essential oil is present in the composition from about 1% by weight to about 10% by weight. In some embodiments, the carrier oil is present in the composition from about 1% by weight to about 5% by weight. In some embodiments, the carrier oil is present in the composition in about 10% by weight, e.g., 10% by weight. In some embodiments, the carrier oil is present in the composition in about 7.5% by weight, e.g., 7.5% by weight or 7.6% by weight. In some embodiments, the carrier oil is present in the composition in about 5% by weight, e.g., 5% by weight. In some embodiments, the carrier oil is present in the composition in about 2.5% by weight, e.g., 2.5% by weight. In some embodiments, the carrier oil is present in the composition in about 1% by weight, e.g., 1% by weight.

### Viscosity Enhancement Agent

The compositions of the present invention can comprise one or more viscosity enhancement agents. A non-limiting example of a viscosity enhancement agent includes silica silylate. The viscosity enhancement agent can also be used to retain the fragrance of a cosmetic composition after application onto a keratinous surface.

In some embodiments, the viscosity enhancement agent is present in the composition from about 0.1% by weight to about 3% by weight. In some embodiments, the viscosity enhancement agent is present in the composition from about 0.1% by weight to about 1% by weight. In some embodiments, the viscosity enhancement agent is present in the composition in about 0.2% by weight, e.g., 0.2% by weight. In some embodiments, the carrier oil is present in the composition in about 7.5% by weight, e.g., 7.5% by weight or 7.6% by weight. In some embodiments, the carrier oil is present in the composition in about 5% by weight, e.g., 5% by weight. In some embodiments, the carrier oil is present in the composition in about 2.5% by weight, e.g., 2.5% by weight. In some embodiments, the carrier oil is present in the composition in about 1% by weight, e.g., 1% by weight.

### Active Ingredients

The formulation may include an active ingredient (or “active”) selected from any personal, healthcare, and pharmaceutical active. As used herein, a “personal care active” means any compound or mixtures of compounds that are known in the art as additives in the personal care formulations that are typically added for treating hair or skin to provide a cosmetic and/or aesthetic benefit. A “healthcare active” means any compound or mixtures of compounds that are known in the art to provide a pharmaceutical or medical benefit. Thus, “healthcare active” includes materials considered as an active ingredient or active drug ingredient as generally used and defined by the United States Department of Health & Human Services Food and Drug Administration, contained in Title 21, Chapter I, of the Code of Federal Regulations, Parts 200-299 and Parts 300-499.

Thus, active ingredient can include any component that is intended to furnish pharmacological activity or other direct effect in the diagnosis, cure, mitigation, treatment, or prevention of disease, or to affect the structure or any function of the body of a human or other animals. The phrase can include those components that may undergo chemical change in the manufacture of drug products and be present in drug products in a modified form intended to furnish the specified activity or effect.

### Cannabinoids

Cannabinoids are a group of chemicals known to activate cannabinoid receptors in cells. The compositions of the present invention can comprise one or more cannabinoids. In some embodiments, the cannabinoid is derived from a naturally occurring source, e.g., a natural oil from a plant. In some embodiments, the cannabinoid is a synthetic cannabinoid, e.g., synthetic cannabidiol. As used herein, a “synthetic cannabinoid” refers to a naturally occurring cannabinoid or a cannabinoid that is not naturally occurring that has been chemically synthesized using methods of synthetic organic chemistry. As used herein, “synthetic cannabidiol” refers to cannabidiol that has been chemically synthesized using methods of synthetic organic chemistry. Synthetic cannabinoids may be synthesized using conventional methods known to one skilled in the art.

In some embodiments, the composition, e.g., an anhydrous gel, comprises a naturally occurring oil comprising a cannabinoid, e.g., an oil derived from a cannabis plant. In some embodiments, the naturally occurring oil is present in the composition from about 1% by weight to about 50% by weight. In some embodiments, the naturally occurring oil is present in the composition from about 1% by weight to about 40% by weight. In some embodiments, the naturally occurring oil is present in the composition from about 1% by weight to about 30% by weight. In some embodiments, the naturally occurring oil is present in the composition from about 1% by weight to about 20% by weight. In some embodiments, the naturally occurring oil is present in the composition from about 1% by weight to about 10% by weight. In some embodiments, the naturally occurring oil is present in the composition from about 1% by weight to about 5% by weight. In some embodiments, the naturally occurring oil is present in the composition in about 10% by weight, e.g., 10% by weight. In some embodiments, the naturally occurring oil is present in the composition in about 5% by weight, e.g., 5% by weight. In some embodiments, the naturally occurring oil is present in the composition in about 2.5% by weight, e.g., 2.5% by weight. In some embodiments, the naturally occurring oil is present in the composition in about 1% by weight, e.g., 1% by weight.

In some embodiments, the composition, e.g., an anhydrous gel, comprises a cannabinoid that is not in a naturally occurring oil, e.g., a synthetic cannabinoid or cannabinoid isolated from a naturally occurring oil. In some embodiments, the cannabinoid is present in the composition from about 1% by weight to about 50% by weight. In some embodiments, the cannabinoid is present in the composition from about 1% by weight to about 40% by weight. In some embodiments, the cannabinoid is present in the composition from about 1% by weight to about 30% by weight. In some embodiments, the cannabinoid is present in the composition from about 1% by weight to about 20% by weight. In some embodiments, the cannabinoid is present in the composition from about 1% by weight to about 10% by weight. In some embodiments, the cannabinoid is present in the composition from about 1% by weight to about 5% by weight. In some embodiments, the cannabinoid is present in the composition in about 10% by weight, e.g., 10% by weight. In some embodiments, the cannabinoid is present in the composition in about 5% by weight, e.g., 5% by weight. In some embodiments, the cannabinoid is present in the composition in about 2.5% by weight, e.g., 2.5% by weight. In some embodiments, the cannabinoid is present in the composition in about 1% by weight, e.g., 1% by weight. In some embodiments, the cannabinoid is present in the composition in about 0.5% by weight, e.g., 0.5% by weight.

The cannabinoids described herein may also be derived from other sources, such as cannabinoids produced by a genetically-modified bacterium. In some embodiments, the cannabinoid is present in the composition from about 1% by weight to about 50% by weight. In some embodiments, the cannabinoid is present in the composition from about 1% by weight to about 40% by weight. In some embodiments, the cannabinoid is present in the composition from about 1% by weight to about 30% by weight. In some embodiments, the cannabinoid is present in the composition from about 1% by weight to about 20% by weight. In some embodiments, the cannabinoid is present in the composition from about 1% by weight to about 10% by weight. In some embodiments, the cannabinoid is present in the composition from about 1% by weight to about 5% by weight. In some embodiments, the cannabinoid is present in the composition in about 10% by weight, e.g., 10% by weight. In some embodiments, the cannabinoid is present in the composition in about 5% by weight, e.g., 5% by weight. In some embodiments, the cannabinoid is present in the composition in about 2.5% by weight, e.g., 2.5% by weight. In some embodiments, the cannabinoid is present in the composition in about 1% by weight, e.g., 1% by weight. In some embodiments, the cannabinoid is present in the composition in about 0.5% by weight, e.g., 0.5% by weight.

“Cannabinoid,” as used herein, is meant to include compounds which interact with the cannabinoid receptor and various cannabinoid mimetics, such as certain tetrahydropyran analogs (e.g., Δ⁹-tetrahydrocannabinol, Δ⁸-tetrahydrocannabinol, 6,6,9-trimethyl-3-pentyl-6H-dibenzo[b,d]pyran-1-ol, 3 -(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ⁶-tetrahydrocannabinol-1,1-dimethylhept-yl, (+)-(3S,4S)-7-hydroxy-Δ⁶-tetrahydrocannabinol-1,1-dimethylh-eptyl, 11-hydroxy-Δ⁹-tetrahydrocannabinol, and Δ⁸-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-meth-yl-4-phenylbutoxy]-1,9-phenanthridinediol 1-acetate)), certain aminoalkylindole analogs (e.g., (R)-(+)-[2,3-dihydro-5-methyl-3-(4-morpholinylmethyl)-pyrrolo[1,2,3-de]-1-,4-benzoxazin-6-yl]-1-naphthalenyl-methanone), certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,-2′,3′,4′,5′,6′-hexahydrobiphenyl), as well as their pharmaceutically acceptable salts, solvates, metabolites (e.g., cutaneous metabolites), and metabolic precursors. Further examples of “cannabinoids” include those compounds described in the references cited below.

“Δ⁹-THC,” as used herein, is meant to refer to Δ⁹-tetrahydrocannabinol as well as to its pharmaceutically acceptable salts, solvates, metabolites (e.g., cutaneous metabolites), and metabolic precursors. Δ⁹-tetrahydrocannabinol is marketed under the generic name “dronabinol.”

“Cannabinol,” as used herein, is meant to refer to 6,6,9-trimethyl-3-pentyl-6H-dibenzo[b,d]pyran-1-ol as well as to pharmaceutically acceptable salts, solvates, metabolites (e.g., cutaneous metabolites), and metabolic precursors of 6,6,9-trimethyl-3-pentyl-6H-dibenzo[b,d]pyran-1-ol. The synthesis of 6,6,9-trimethyl-3-pentyl-6H-dibenzo[b,d]pyran-1-ol is described in, for example, Novak et al., Tetrahedron Letters, 23:253 (1982), which is hereby incorporated by reference.

“Cannabidiol,” as used herein, is meant to refer to 2-[3-methyl-6-(1-methylethenyl)-2-cyclohexen-1-yl]-5-pentyl-1,3-benzenediol as well as to pharmaceutically acceptable salts, solvates, metabolites (e.g., cutaneous metabolites), and metabolic precursors of 2-[3-methyl-6-(1-methylethenyl)-2-cyclohexen-1-yl]-5-pentyl-1,3-benzenediol. The synthesis of 2-[3-methyl-6-(1-methylethenyl)-2-cyclohexen-1-yl]-5-pentyl-1,3-benzenediol is described, for example, in Petilka et al., Hely. Chim. Acta, 52:1102 (1969) and in Mechoulam et al., J. Am. Chem. Soc., 87:3273 (1965), which are hereby incorporated by reference.

“Nabilone,” as used herein, is meant to refer to 3-(1,1-dimethylheptyl)-6,6a,7,8,10,10a-hexahydro-1-hydroxy-6,6-dimethyl-9-H-dibenzo[b,d]pyran-9-one as well as to pharmaceutically acceptable salts, solvates, metabolites (e.g., cutaneous metabolites), and metabolic precursors of 3-(1,1-dimethylheptyl)-6,6a,7,8,10,10a-hexahydro-1-hydroxy-6,6-dimethyl-9H-dibenzo[b,d]pyran-9-one. 3-(1,1-dimethylheptyl)-6,6a,7,8,10,10a-hexahydro-1-hydroxy-6,6-dimethyl-9-H-dibenzo[b,d]pyran-9-one is approved for use in the United Kingdom for treating nausea and vomiting associated with chemotherapy, and its preparation is described, for example, in U.S. Pat. No. 3,968,125 to Archer, which is hereby incorporated by reference.

“Levonantradol,” as used herein, is meant to refer to (−)-(6S,6aR,9R,10aR)-5,6,6a,7,8,9,10,10a-octahydro-6-methyl-3-[(R)-1-meth-yl-4-phenylbutoxy]-1,9-phenanthridinediol 1-acetate, as well as to pharmaceutically acceptable salts, solvates, metabolites (e.g., cutaneous metabolites), and metabolic precursors of (−)-(6S,6aR,9R,10aR)-5,6,6a,7,8,9,10,10a-octahydro-6-methyl -3-[(R)-1-methyl -4-phenylbutoxy]-1,9-phenanthridinediol 1-acetate. (−)-(6S,6aR,9R,10aR)-5,6,6a,7,8,9,10,10a-octahydro-6-methyl-3-[(R)-1-methyl-4-phenylbutoxy]-1,9-phenanthridinediol 1-acetate is particularly useful in pain control, and its synthesis is described in Belgian Pat. No. 854,655, which is hereby incorporated by reference; in U.S. Pat. Nos. 4,206,225, 4,232,018, and 4,260,764, each to Johnson, which are hereby incorporated by reference; in U.S. Pat. No. 4,235,913 to Johnson et al., which is hereby incorporated by reference; in U.S. Pat. No. 4,243,674 to Bindra, which is hereby incorporated by reference; and in U.S. Pat. Nos. 4,263,438, 4,270,005, and 4,283,569, each to Althuis et al., which are hereby incorporated by reference.

“(−)-HU-210,” as used herein, is meant to refer to (−)-(3S,4S)-7-hydroxy-Δ⁶-tetrahydrocannabinol-1,1-dimethylheptyl as well as to pharmaceutically acceptable salts, solvates, metabolites (e.g., cutaneous metabolites), and metabolic precursors of (−)-(3S,4S)-7-hydroxy-Δ⁶-tetrahydrocannabinol-1,1-dimethylheptyl. (−)-(3S,4S)-7-hydroxy-Δ⁶-tetrahydrocannabinol-1,1-dimethylheptyl is particularly useful in pain control, and its preparation is described in U.S. Pat. Nos. 4,876,276 and 5,521,215, each to Mechoulam et al., which are hereby incorporated by reference.

“(+)-HU-210,” as used herein, is meant to refer to (+)-(3S,4S)-7-hydroxy-Δ⁶-tetrahydrocannabinol-1,1-dimethylheptyl as well as to pharmaceutically acceptable salts, solvates, metabolites (e.g., cutaneous metabolites), and metabolic precursors of (+)-(3S,4S)-7-hydroxy-Δ⁶-tetrahydrocannabinol-1,1-dimethylheptyl. (+)-(3S,4S)-7-hydroxy-Δ⁹-tetrahydrocannabinol-1,1-dimethylheptyl is sometimes referred to as HU-211 and/or dexanabinol; it is an antagonist of the N-methyl-D-aspartate receptor; and its preparation is described in U.S. Pat. Nos. 4,876,276 and 5,521,215, each to Mechoulam et al., which are hereby incorporated by reference.

“11-hydroxy-Δ⁹-THC,” as used herein is meant to refer to 11-hydroxy-Δ⁹-tetrahydrocannabinol as well as to its pharmaceutically acceptable salts, solvates, metabolites (e.g., cutaneous metabolites), and metabolic precursors. 11-hydroxy-Δ⁹-tetrahydrocannabinol is a more hydrophilic, psychoactive metabolite of Δ⁹-tetrahydrocannabinol, and its laboratory synthesis has been described in Siegel et al., J. Org. Chem., 54:5428 (1989), which is hereby incorporated by reference.

“Δ⁸-THC-11-oic acid,” as used herein, is meant to refer to Δ⁸-tetrahydrocannabinol-11-oic acid, as well as to its pharmaceutically acceptable salts, solvates, metabolites (e.g., cutaneous metabolites), and metabolic precursors. Δ⁸-tetrahydrocannabinol-11-oic acid is a naturally occurring derivative of 6a,7,10,10a-tetrahydro-6,6,9-trimethyl-3-pentyl-6H-dibenzo[b,d]pyran-1-ol (which is a minor component of Cannabis sativa) and is produced from 6a,7, 10,10a-tetrahydro-6,6,9-trimethyl-3-pentyl-6H-dibenzo[b,d]pyran-1-o-1 via a series of biotransformations mediated primarily by mammalian liver enzymes. Δ⁸-tetrahydrocannabinol-11-oic acid can also be produced synthetically by reference to the synthetic schemes set forth in U.S. Pat. No. 6,162,829 to Burstein, which is hereby incorporated by reference. Δ⁸-tetrahydrocannabin-ol-11-oic acid is more hydrophilic than 6a,7,10,10a-tetrahydro-6,6,9-trimethyl-3-pentyl-6H-dibenzo[b,d]pyran-1-ol-, and it has analgesic activity.

“CP 55,940,” as used herein, refers to 4-(1,1-dimethylheptyl)-2,3′-dihydroxy-6′alpha-(3-hydroxypropyl)-1′,2′,3′,-4′,5′,6′-hexahydrobiphenyl, as well as to its pharmaceutically acceptable salts, solvates, metabolites (e.g., cutaneous metabolites), and metabolic precursors. 4-(1,1-dimethylheptyl)-2,3′-dihydroxy-6′alpha-(3-hydroxypropyl)-1′,2′,3′,-4′,5′,6′-hexahydrobiphenyl is sometimes referred to as (−)-cis-3-[2-Hydroxy-4-(1,1-dimethylheptyl)phenyl]-trans-4-(3-hydroxypropyl)cyclohexanol, and it is commercially available from Tocris Cookson, Inc., Ellisville, Mo. Its preparation has been described in U.S. Pat. No. 4,371,720 to Johnson et al. and U.S. Pat. No. 4,663,474 to Urban, which are hereby incorporated by reference.

“R(+)-WIN 55,212-2,” as used herein, refers to (R)-(+)-[2,3-dihydro-5-methyl-3-(4-morpholinylmethyl)pyrrolo[1,2,3-de]-1,4-benzoxazin-6-yl]-1-naphthalenylmethanone, as well as to its pharmaceutically acceptable salts, solvates, metabolites (e.g., cutaneous metabolites), and metabolic precursors. (R)-(+)-[2,3-dihydro-5-methyl-3-(4-morpholinylmethyl)-pyrrolo[1,2,3-de]-1-,4-benzoxazin-6-yl]-1-naphthalenyl-methanone (in its mesylate form) is commercially available, for example, from Tocris Cookson, Inc., Ellisville, Mo., and from Research Biochemicals International, Natick, Mass.

The compositions described herein can further include one or more additional cannabinoids. The one or more additional cannabinoids can be selected from the aforementioned list of cannabinoids or it (they) can be selected from cannabinoids which are not contained in the aforementioned list, such as Δ⁸-THC, high affinity cannabinoid receptor agonists (other than R(+)-WIN 55,212-2 and CP 55,940), and the like. Illustratively, the cannabinoid composition can include two or more cannabinoids, each being selected from the group consisting of Δ⁹-THC, cannabinol, cannabidiol, nabilone, levonantradol, (−)-HU-210, (+)-HU-210, 11-hydroxyΔ⁹-THC, Δ⁸-THC-11-oic acid, CP 55,940, and R(+)-WIN 55,212-2.

“Metabolic precursors” of cannabinoids, as used herein, are meant to include prodrugs and other materials that are metabolized in the subject's body (e.g., cutaneously or systemically or both) to a cannabinoid or an active cannabinoid mimetic. Suitable metabolic precursors include those that are less lipophilic (i.e., more water soluble) relative to the cannabinoid into which they are metabolized. Examples of such metabolic precursors include those described in, for example, U.S. Pat. No. 5,847,128 to Martin et al., which is hereby incorporated by reference.

“Metabolites” of cannabinoids, as used herein, are meant to include compounds which are produced by the metabolic processes (e.g., cutaneous metabolic processes and/or systemic metabolic processes) of the subject's body. Suitable metabolites can be identified, for example, by studying the kinetics of drug enzymatic metabolism in skin homogenates. Illustratively, skin homogenates can be prepared from 250-μm dermatomed fresh healthy abdominal plastic surgery samples. The skin is homogenized (e.g., using a Polytron tissue homogenizer and ground glass homogenizer fitted with a glass pestle) in 4-(2-hydroxyethyl)-1-piperazineethanesulfonic acid (“HEPES”)-buffered Hanks' balanced salt solution. Whole homogenates can be used for these studies or, if significant mitochondrial or nuclear metabolism is found not to occur (e.g., by comparing the degree of metabolism in the supernatant to the degree of metabolism in the whole homogenate), the studies can be carried out on only the supernatant fraction. The drug (solubilized in, for example, buffer, ethanol, dimethylsulfoxide, or combinations thereof) is then incubated with the homogenate (or supernatant) along with NADPH (or a generating system), NADH, MgCl₂, and bovine serum albumin. The total volume of ethanol in the reaction mixture should be small (e.g., under 2%) to help minimize ethanol's detrimental effects on the enzymes. After incubating for a period of time, the reaction is terminated with 15% trichloroacetic acid, and the drug and its metabolites are obtained by solid-phase extraction. The metabolite or metabolites formed can then be identified and assayed by any suitable method (e.g., HPLC).

### Occlusivity Agent

The formulation may include an occlusivity agent configured to provide occlusivity when the formulation is applied on top of the skin. The occlusivity agent may include petrolatum, organic wax, silicone wax, polyacrylates and methacrylates (exemplified by, but not limited to Eudragit® E100, S100, L100, and L100-55), polyvinyl pyrolidone, polyvinyl alcohol, vinylacetate-vinylpyrolidone copolymer, or any combination thereof. A majority of film-forming polymers can be considered to provide occlusive properties to the formulation and thus any suitable film-forming polymer may be used in the present formulation.

The occlusivity agent may be a wax or a wax-like material. The waxes or wax-like materials useful in the formulation according to the present disclosure generally have a melting point range of about 35 to 120° C. at atmospheric pressure. Waxes in this category include synthetic wax, ceresin, paraffin, ozokerite, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, or mixtures thereof. Additionally, the occlusivity agent may include waxes capable of being used as non-silicone fatty substances, animal waxes, such as beeswax; vegetable waxes, such as carnauba, candelilla wax; mineral waxes, such as paraffin or lignite wax; microcrystalline waxes; ozokerites; synthetic waxes, including polyethylene waxes, and waxes obtained by the Fischer-Tropsch synthesis. Additionally, the occlusivity agent may include silicone waxes, polymethylsiloxane alkyls, alkoxys and/or esters.

### Additional Optional Components

The formulation may also contain a number of optional ingredients. In particular, these optional components are selected from those known in the art to be ingredients used in personal care or pharmaceutical formulations. Illustrative, non-limiting examples include surfactants, solvents, powders, coloring agents, thickeners, waxes, gelling agents or clays, stabilizing agents, pH regulators, silicones, or other suitable agents.

A thickening agent may be added to provide a desired or convenient viscosity. For example, viscosities within the range of 500 to 25,000 mm²/s at 25° C. Alternatively, thickening agents may be added to obtain viscosities within the range of about 3,000 to about 7,000 mm²/s. Suitable thickening agents are exemplified by sodium alginate, gum arable, polyoxyethylene, guar gum, hydroxypropyl guar gum, ethoxylated alcohols, such as laureth-4 or polyethylene glycol 400, cellulose derivatives exemplified by methylcellulose, methylhydroxypropylcellulose, hydroxypropylcellulose, polypropylhydroxyethylcellulose, starch, and starch derivatives exemplified by hydroxyethylamylose and starch amylose, locust bean gum, electrolytes exemplified by sodium chloride and ammonium chloride, and saccharides such as fructose and glucose, and derivatives of saccharides such as PEG-120 methyl glucose diolate or mixtures of 2 or more of these. Alternatively the thickening agent is selected from cellulose derivatives, saccharide derivatives, and electrolytes, or from a combination of two or more of the above thickening agents exemplified by a combination of a cellulose derivative and any electrolyte, and a starch derivative and any electrolyte. The thickening agent may be present in an amount from about 0.05 to about 10% by weight, or, alternatively about 0.05 to about 5% by weight based on the total weight of the formulation.

Also, various cosmetic, personal care, and cosmetic components may be included aside from the excipient or excipients. Examples of suitable cosmetic, and personal care components include, but are not limited to, alcohols, fatty alcohols and polyols, aldehydes, alkanolamines, alkoxylated alcohols butylene copolymers, carbohydrates (e.g. polysaccharides, chitosan and derivatives), carboxylic acids, carbomers, esters, ethers and polymeric ethers (e.g. PEG derivatives, PPG derivatives), glyceryl esters and derivatives, halogen compounds, heterocyclic compounds including salts, hydrophilic colloids and derivatives including salts and gums (e.g. cellulose derivatives, gelatin, xanthan gum, natural gums), imidazolines, inorganic materials (clay, TiO₂, ZnO), ketones (e.g. camphor), isethionates, lanolin and derivatives, organic salts, phenols including salts phosphorus compounds (e.g. phosphate derivatives), polyacrylates and acrylate copolymers, synthetic polymers including salts, siloxanes and silanes, sorbitan derivatives, sterols, sulfonic acids and derivatives and waxes.

Other additives can include powders and pigments. The powder component that may be included can be generally defined as dry, particulate matter having an average particle size of about 0.02-50 microns. The particulate matter may be colored or non-colored (for example, white). Suitable powders include, but are not limited to, bismuth oxychloride, titanated mica, fumed silica, spherical silica beads, polymethylmethacrylate beads. The above mentioned powders may be surface treated to render the particles hydrophobic in nature.

The powder component also may also include various organic and inorganic pigments. The organic pigments are generally various aromatic types including azo, indigoid, triphenylmethane, anthraquinone, and xanthine dyes. Inorganic pigments generally consist of insoluble metallic salts of certified color additives, referred to as the Lakes or iron oxides. A pulverulent coloring agent, such as carbon black, and titanium dioxide, pearlescent agents, generally used as a mixture with colored pigments, or some organic dyes, generally used as a mixture with colored pigments and commonly used in the cosmetics industry, can be added to the formulation. In general, these coloring agents can be present in an amount by weight from about 0 to 20% with respect to the weight of the final formulation.

Pulverulent inorganic or organic fillers can also be added, generally in an amount by weight from about 0 to about 40% with respect to the weight of the final formulation. These pulverulent fillers can be chosen from talc, micas, kaolin, zinc or titanium oxides, calcium or magnesium carbonates, silica, spherical titanium dioxide, glass or ceramic beads, metal soaps derived from carboxylic acids having 8-22 carbon atoms, non-expanded synthetic polymer powders, expanded powders and powders from natural organic compounds, such as cereal starches, which may or may not be crosslinked, copolymer microspheres, polytrap, and silicone resin microbeads.

Optional components included in the present formulation may also include other silicones (including any already described above), organofunctional siloxanes, alkylmethylsiloxanes, siloxane resins and silicone gums.

The topical formulations according to the present disclosure may be in the form of a cream, a gel, a powder, a paste, or a freely pourable liquid. Generally, such formulations can generally be prepared at room temperature if no solid materials at room temperature are presents in the formulations, using simple propeller mixers, Brookfield counter-rotating mixers, or homogenizing mixers. No special equipment or processing conditions are typically required. Depending on the type of form made, the method of preparation will be different, but such methods are well known by those of ordinary skill in the art.

Anhydrous formulations may be prepared without the addition of any preservatives.

In embodiments where the substrate is skin, the formulation is applied to the skin. The skin may be healthy and intact, or it may be damaged or wounded. The formulation may be applied, i.e., rubbed or coated, directly onto the skin. Alternatively, the formulation may be deposited on a transdermal patch prior to application of the formulation to the substrate, i.e., to the skin.

The formulations according to the present disclosure can be used by standard and well-known methods, such as applying them to the human body, e.g. skin, hair, or teeth, using applicators, brushes, applying by hand, pouring them and/or possibly rubbing or massaging the formulation onto or into the body. Removal methods are also well known standard methods, including washing, wiping, peeling and the like. According to some embodiments, no removal of the formulation is required as the formulation is fully absorbed into the skin, such that no residue remains on the skin. An effective amount of the formulation for the particular purpose is applied to the skin. Such effective or therapeutic amounts generally range from about 1 mg/cm² to about 10 mg/cm². Application to the skin typically includes working the formulation into the skin. This method for applying to the skin comprises the steps of contacting the skin with the formulation in an effective amount and then rubbing the formulation onto the skin. These steps can be repeated as many times as desired to achieve the desired benefit.

### EXAMPLES

The compositions of the present invention may be prepared using methodology that is well known by an artisan of ordinary skill, e.g., by using established mixing/blending procedures, whereby the ingredient(s) to be incorporated are simply added to the composition, e.g., the anhydrous gel.

The following is a description of the manufacture of specific gels and compositions of the present invention. These examples are non-limiting and other gels and compositions of the invention can be prepared in an analogous manner by a person of ordinary skill in the art.

### Example 1

### Anhydrous Cosmetic Base Gel A

TABLE 1

| Ingredient | Weight % |
|---|---|
| Isododecane (and) dimethicone/bis- | 67.40 |
| isobutyl PPG-20 crosspolymer (85:15) | |
| Caprylic/capric triglyceride | 8.50 |
| Phenyl trimethicone | 8.30 |
| Chrystaphyl ® (lauryl lactate) | 0.25 |
| Sunflower seed oil | 15.00 |
| Silica silylate | 0.55 |
| | 100.00 |

### Example 2

### Anhydrous Cosmetic Base Gel B

TABLE 2

| Ingredient | Weight % |
|---|---|
| Isododecane (and) dimethicone/bis- | 65.00 |
| isobutyl PPG-20 crosspolymer (85:15) | |
| Caprylic/capric triglyceride | 8.50 |
| Phenyl trimethicone | 8.30 |
| Chrystaphyl ® (lauryl lactate) | 1.00 |
| Myristly lactate | 2.00 |
| Sunflower seed oil | 14.90 |
| Silica silylate | 0.30 |
| | 100.00 |

### Example 3

### Anhydrous Cosmetic Base Gel C

TABLE 3

| Ingredient | Weight % |
|---|---|
| Isododecane (and) dimethicone/bis- | 65.00 |
| isobutyl PPG-20 crosspolymer (85:15) | |
| Caprylic/capric triglyceride | 8.50 |
| Phenyl trimethicone | 8.30 |
| Chrystaphyl ® (lauryl lactate) | 0.25 |
| Myristyl lactate | 1.75 |
| Cetyl lactate | 3.00 |
| Sunflower seed oil | 12.90 |
| Silica silylate | 0.30 |
| | 100.00 |

### Example 4

### Anhydrous Cosmetic Base Gel D

TABLE 4

| Ingredient | Weight % |
|---|---|
| Isododecane (and) dimethicone/bis- | 55.00 |
| isobutyl PPG-20 crosspolymer (85:15) | |
| Dimethicone (and) dimethicone/vinyl | 10.00 |
| dimethicone crosspolymer (and) | |
| dimethicone crosspolymer (and) beeswax | |
| (and) silica (and) silica silylate | |
| Caprylic/capric triglyceride | 6.50 |
| Coco-caprylate/caprate | 2.00 |
| Phenyl trimethicone | 8.30 |
| Chrystaphyl ® (lauryl lactate) | 2.00 |
| Myristyl lactate | 3.00 |
| Sunflower seed oil | 12.90 |
| Silica silylate | 0.30 |

### Example 5

### Anhydrous Cosmetic Base Gel E

TABLE 5

| Ingredient | Weight % |
|---|---|
| Isododecane (and) dimethicone/bis- | 65.00 |
| isobutyl PPG-20 crosspolymer (85:15) | |
| Caprylic/capric triglyceride | 8.50 |
| Phenyl trimethicone | 8.30 |
| Chrystaphyl ® (lauryl lactate) | 0.25 |
| Camphor | 5.00 |
| Sunflower seed oil | 12.65 |
| Silica silylate | 0.30 |
| | 100.00 |

### Example 6

### Anhydrous Cosmetic Base F with Birch Oil

TABLE 6

| Ingredient | Weight % |
|---|---|
| Isododecane (and) dimethicone/bis- | 67.00 |
| isobutyl PPG-20 crosspolymer (85:15) | |
| Caprylic/capric triglyceride | 8.50 |
| Phenyl trimethicone | 8.25 |
| Chrystaphyl ® (lauryl lactate) | 0.25 |
| Myristyl lactate | 2.75 |
| Betula lenta (bark) oil | 5.00 |
| Sunflower seed oil | 8.00 |
| Silica silylate | 0.25 |
| | 100.00 |

### Example 7

### Anhydrous Mentholated Gel (Compare to Vicks VapoRub)

TABLE 7

| Ingredient | Weight % |
|---|---|
| Anhydrous Camphorated Base E | 93.80 |
| Peppermint oil | 5.00 |
| Eucalyptus oil | 1.20 |

### Example 8

### Cannabidiol (CBD) Oil-Containing Gel

TABLE 8

| Ingredient | Weight % |
|---|---|
| Anhydrous Cosmetic Base Gel B | 94.50 |
| CBD oil | 5.00 |
| Fragrance | 0.50 |

### Example 9

### Cannabis Oil-Containing Gel

TABLE 9

| Ingredient | Weight % |
|---|---|
| Anhydrous Cosmetic Base Gel F | 95.00 |
| Cannabis oil | 5.00 |
| | 100.00 |

### Other Embodiments

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Detailed Description, but rather is as set forth in the appended claims. Those of ordinary skill in the art will appreciate that various changes and modifications to this description may be made without departing from the spirit or scope of the present invention, as defined in the following claims.

What is claimed is:

1. A composition comprising a silicone-based excipient, a carrier oil, an emollient, and a viscosity enhancement agent, wherein the composition does not comprise a healthcare active.

2. The composition of claim 1, wherein the silicone-based excipient is present in the composition in at least about 80% by weight.

3. The composition of claim 1, wherein the silicone-based excipient is provided in a carrier fluid.

4. The composition of claim 3, wherein the carrier fluid is isododecane.

5. The composition of claim 3, wherein the weight ratio of carrier fluid to silicone-based excipient is about 85:15.

6. The composition of claim 1, wherein the carrier oil is a natural oil.

7. The composition of claim 1, wherein the carrier oil is selected from the group consisting of sunflower seed oil, coconut oil, grape seed oil, olive oil, jojoba, and combinations thereof.

8. The composition of claim 1, wherein the carrier oil is sunflower seed oil.

9. The composition of claim 1, wherein the carrier oil is present in the composition in at least about 5%.

10. The composition of claim 1, wherein the weight ratio of silicone-based excipient to carrier oil is from about to about 1:0.2 to about 2:1.

11. The composition of claim 1, wherein the weight ratio of silicone-based excipient to carrier oil is about 1.5:0.5.

12. The composition of claim 1, further comprising an emollient.

13. The composition of claim 12, wherein the emollient is an ester of a fatty acid.

14. The composition of claim 12, wherein the emollient is a triglyceride.

15. The composition of claim 1, wherein the composition further comprises a silicone fluid.

16. The composition of claim 15, wherein the silicone fluid is a trimethicone.

17. The composition of claim 16, wherein the trimethicone is phenyl trimethicone.

18. The composition of claim 1, wherein the composition is an anhydrous gel.

19. The composition of claim 1, wherein the composition is formulated for topical administration.

20. The composition of claim 1, wherein the viscosity enhancement agent is silica silylate.
